(19) United States
(12) Patent Application Publication
(10) Pub. No.: US 2019/0225932 A1
(43) Pub. Date: Jul. 25, 2019

(54) CULTIVATION AND ENERGY EFFICIENT HARVESTING OF MICROALGAE USING THEREMOREVERSIBLE SOL-GEL TRANSITION

(71) Applicants: Radhakrishna Sureshkumar, Jamesville, NY (US); Bendy Estime, Jacksonville, FL (US); Dacheng Ren, Syracuse, NY (US)

(72) Inventors: Radhakrishna Sureshkumar, Jamesville, NY (US); Bendy Estime, Jacksonville, FL (US); Dacheng Ren, Syracuse, NY (US)

(73) Assignee: SYRACUSE UNIVERSITY, SYRACUSE, NY (US)

(21) Appl. No.: 16/307,322

(22) PCT Filed: Jun. 6, 2017
(86) PCT No.: PCT/US17/36050

§ 371 Date: Dec. 5, 2018

Related U.S. Application Data
(60) Provisional application No. 62/347,282, filed on Jun. 8, 2016.

(51) Int. Cl. C12N 1/12

(52) U.S. Cl. CPC C12N 1/12; C12N 2500/60; C12N 2511/00; C12N 2539/00; C12N 2513/00; C12N 2523/00

(57) ABSTRACT

A Tris-Acetate-Phosphate-Pluronic (TAPP) medium that undergoes thermoreversible sol-gel transitions to efficiently culture and harvest microalgae without affecting productivity. After seeding microalgae in a TAPP medium in solution phase at 15 degrees C., the temperature is increased by 7 degrees C. to induce gelation. Within the gel, microalgae grow in large clusters rather than as isolated cells. Such clusters are easily harvested gravimetrically by decreasing the temperature to bring the medium to a solution phase. The settling velocity of the microalgal clusters is approximately ten times larger than that of individual cells cultured in typical solution media. Hence, microalgae can be cultured without constant mixing and about 90 percent of the biomass can be harvested in an energy efficient fashion.

### CROSS-REFERENCE TO RELATED APPLICATIONS

This application claims priority to U.S. Provisional No. 62/347,282, filed on Jun. 8, 2016.

### BACKGROUND OF THE INVENTION

### 1. Field of the Invention

The present invention relates to cell culturing and, more specifically, to the use of thermoreversible sol-gel transitions to culture and harvest cell cultures without the need for energy intensive mixing and centrifuging.

### 2. Description of the Related Art

The development of methods for high throughput cultivation and efficient harvesting of microalgae has, over the past decades, constituted an active field of research. Despite major advances, there is still a need to optimize and increase productivity in microalgal cultivation systems in order to make microalgal biofuels production a more viable option. It is also imperative to improve microalgal harvesting processes which currently account for about thirty percent of total production cost.

Many cultivation methods have been proposed to improve microalgal biomass production. For instance, growth medium modifications with high salt and nutrient deprivation have been used to enhance accumulation of specific chemicals such as lipids and carbohydrates. Furthermore, biofilm and biofouling of microalgae that are often portrayed as challenges for suspended culture have recently been explored as cultivation methods for large-scale microalgal biomass production. Among many others, the large decrease in water consumption and the simplification of the harvesting process are considered as two major benefits of biofilm cultivation of microalgae. As for suspended cultivation, constant mixing is usually necessary during the entire cultivation period and the current harvesting methods often involving centrifugation, pumping or electrophoresis techniques are largely energy intensive. The alternatives that have been proposed thus far are yet to resolve the energy consumption issue.

Pluronic is an amphiphilic ABA type copolymer composed of both hydrophobic Polypropylene Oxide (PPO) block parts and hydrophilic Polyethylene Oxide (PEO) block parts known for good biocompatibility and low toxicity. The applications of this copolymer are highly diversified. For example, the copolymer pluronic F-127 is believed to be a good carrier for most routes in drug administration and is therefore valuable in pharmaceutical formulations. Pluronic has also largely been suggested for its potential in controlling biofouling. Moreover, this copolymer is well known for its effectiveness in producing stable surface patterns and can be useful in long term single-cell culture.

### BRIEF SUMMARY OF THE INVENTION

The present invention provide a culture medium comprising a Tris-Acetate-Phosphate (TAPP) solution and an amount of pluronic dissolved in the Tris-Acetate-Phosphate solution to form a medium capable of a sol to gel transition. The amount of pluronic dissolved in the Tris-Acetate-Phosphate solution results in a concentration of pluronic of at least 18 percent by weight, at least 20 percent by weight, or at least 22 percent by weight. The chemical composition of the pluronic is PEO₁₀₀PPO₆₅PEO₁₀₀ and the total molecular weight is 12600 g mol⁻¹. The pluronic has a ratio of PEO to PPO of 2:1 by weight.

The present invention also provides a method of culturing an organism, comprising the steps of providing a culture medium comprising a Tris-Acetate-Phosphate solution and an amount of pluronic dissolved in the Tris-Acetate-Phosphate solution, maintaining the culture medium at a first temperature where the culture medium is in a sol state, seeding the culture medium with an organism to be cultured, heating the culture medium to a second temperature where the culture medium is in a gel state, and allowing the organism to grow while the culture medium is in the gel state. The method may further comprise the step of cooling the culture medium to a third temperature where the culture medium is in a sol state. The method may further comprise the step of allowing the organism to settle with the culture medium is in a sol state. The method may further comprise the step of heating the culture medium to a fourth temperature where the culture medium is in a sol state. The method may further comprise the step of harvesting the organism that has settled from the culture medium while it is in a gel state. The first temperature is below a sol to gel transition temperature of the culture medium. The second temperature is above the sol-gel transition temperature of the culture medium. The third temperature is below the sol-gel transition temperature of the culture medium. The fourth temperature is above the sol-gel transition temperature of the culture medium.

The present invention thus provides energy efficient microalgal cultivation and harvesting using a microalgal cultivation and harvesting strategy that employs the thermoreversible copolymer pluronic. In particular, a Tris-Acetate-Phosphate-Pluronic medium was used and undergoes thermoreversible sol-gel transitions to efficiently culture and harvest microalgae without affecting productivity. With the copolymer pluronic, the gelation process is completely reversible upon cooling. Gelation points of pluronic F-127 aqueous solutions are often between 15° C. to 30° C. This intersects with the temperature range often involved in microalgal cultivation. After seeding microalgae in a TAPP medium in solution phase at 15 degrees C., the temperature is increased by 7 degrees C. to induce gelation. Within the gel, microalgae grow in large clusters rather than as isolated cells. Such clusters are easily harvested gravimetrically by decreasing the temperature to bring the medium to a solution phase. The settling velocity of the microalgal clusters is approximately ten times larger than that of individual cells cultured in typical solution media. Hence, microalgae can be cultured without constant mixing and about 90 percent of the biomass can be harvested in an energy efficient fashion.

The thermorheological properties of the pluronic-based medium as well as the resulting pluronic-microalgae matrix after cultivation were systematically characterized. Cultivation experiments were performed using microalga Chlamydomonas reinhardtii and microalgal biomass production in the TAPP medium were assessed both qualitatively and quantitatively. Thus, the present invention provides a framework to efficiently harvest the microalgal biomass produced with small variations of temperature.

### BRIEF DESCRIPTION OF THE SEVERAL VIEWS OF THE DRAWING(S)

The present invention will be more fully understood and appreciated by reading the following Detailed Description in conjunction with the accompanying drawings, in which:

FIG. 1 is a schematic of microalgal cultivation and harvesting process using thermoreversible sol-gel transition. Microalgal cells are seeded in TAPP medium in solution at 15° C. Then, the temperature is raised at 22° C. for gelation and trapped microalgal cultivation. After the cultivation period, the temperature is decreased to 15° C. allowing microalgal clusters to settle at the bottom. The temperature is finally raised to 25° C. and microalgal clusters are scraped off the TAPP surface;

FIG. 2 is a series of graphs of thermorheological properties of the TAPP medium, where: (a) Storage modulus (circle) and loss modulus (star) of the 22% pluronic in TAPP sample as a function of temperature, (b) viscosity profile of the 22% in TAPP pluronic sample as a function of temperature, (c) Storage modulus (circle) and loss modulus (star) of the 18% pluronic in TAPP sample as a function of temperature and (d) viscosity profile of the 18% pluronic in TAPP sample as a function of temperature; and

FIG. 3 is a series of graphs of the microalgal biomass production in the TAPP medium. (a) Microalgal biomass generation (g/l), (b) weight percentage of lipid and carbohydrate in microalgal biomass and (c) microalgal cell diameter under TAP medium, 18% pluronic, 20% pluronic and 22% pluronic TAPP growth conditions. Bars represent means of 30 measurements and error bars are one S.D.;

FIG. 4 is a series of graphs of the effects of microalgal proliferation on the thermorheological behavior of the TAPP medium. (a) Storage modulus (circle) and loss modulus (star) of the 22% pluronic in TAPP sample, with (blue) and without (red) microalgae, as a function of temperature, (b) viscosity profile of the 22% pluronic in TAPP sample, with (blue) and without (red) microalgae, as a function of temperature, (c) Storage modulus (circle) and loss modulus (star) of the 18% pluronic in TAPP sample, with (blue) and without (red) microalgae, as a function of temperature, (d) viscosity profile of the 18% pluronic in TAPP sample, with (blue) and without (red) microalgae, as a function of temperature;

FIG. 5 is a series of images of the characterization of microalgal settling. (a) Percentage of microalgal biomass recovery through settling determined through results from optical density measurements at regular intervals. Data points represent means of 30 measurements and error bars are one S.D. (b) Image of microalgal cells in TAP medium and (c) image of microalgal cell clusters in TAPP medium. Scale bars are 50 μm.

### DETAILED DESCRIPTION OF THE INVENTION

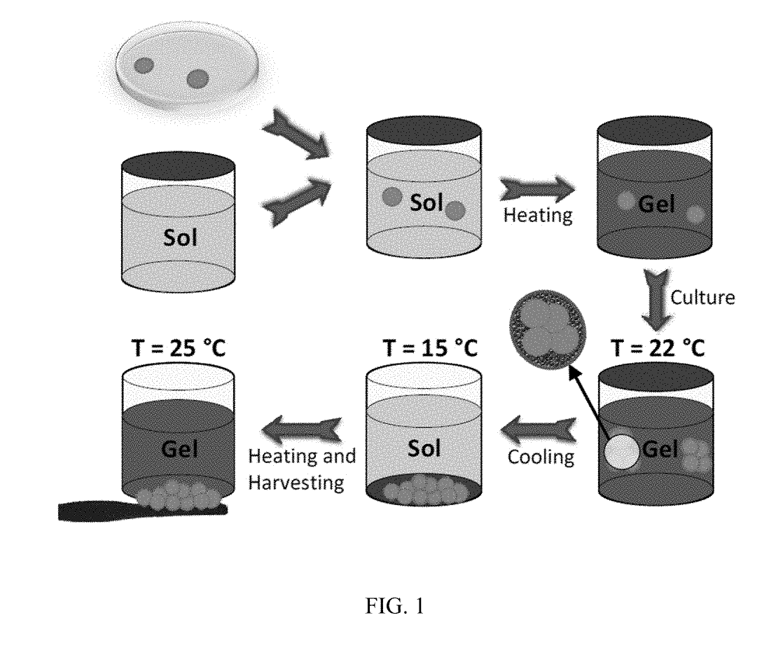

Referring to the figures, wherein like numerals refer to like parts throughout, there is seen in FIG. 1 a Tris-Acetate-Phosphate-Pluronic medium with thermoreversible sol-gel transition properties that was developed for energy efficient cultivation and harvesting of microalgae. The thermorheological properties of the medium and the effects of microalgal proliferation on such properties were experimentally characterized to offer a framework for designing of robust microalgal cultivation and harvesting systems using the thermoreversible copolymer pluronic. Microalga Chlamydomonas reinhardtii was successfully cultivated in the TAPP medium and led to production of microalgal biomass with similar productivity, lipid and carbohydrate composition than that obtained from cultivation in the traditional TAP medium. The harvesting process of the microalgal biomass produced was highly simplified in the system therein. In fact, confinement of microalgal cells in the pluronic matrix led to a cluster distribution that increases the settling velocity by a factor of ten. Through small variations of temperature, microalgal clusters were allowed to settle and harvesting of microalgae simply involved scraping the microalgal clusters off the surface after a subsequent jellification of the broth. These findings confirm that microalgae can efficiently be cultured in the TAPP system which does not require constant mixing like typical solution broths. The present invention is thus important not only for proposing a system that efficiently harvests about ninety percent of the microalgal biomass produced through a simple process, but also for confirming biocompatibility of pluronic with microalgae. Studies to continue this work may include recultivation of the non-harvested microalgae for a sustainable use of pluronic and potential functionalization of the copolymer in order to minimize the concentration necessary to confer the required properties. Other studies may also be undertaken to elucidate the interactions of different microalgal species and strains with the pluronic matrix and to further characterize the microalgal clusters obtained through confinement in the matrix.

### Example

In order to obtain a range of pluronic concentrations that can confer the suitable properties necessary for the proposed thermoreversible microalgal cultivation and harvesting system, TAPP media with different pluronic concentrations were prepared and were subjected to rheological testing. First, strain sweep measurements were performed on the TAPP media samples in order to determine the linear viscoelastic region necessary for the succeeding analyses. The linear viscoelastic regions varied depending on the concentration of pluronic. Nonetheless, it was found that for all the samples and under the operating conditions, a 0.5% strain at a frequency of 1 Hz was favorable for analyses in the linear viscoelastic region.

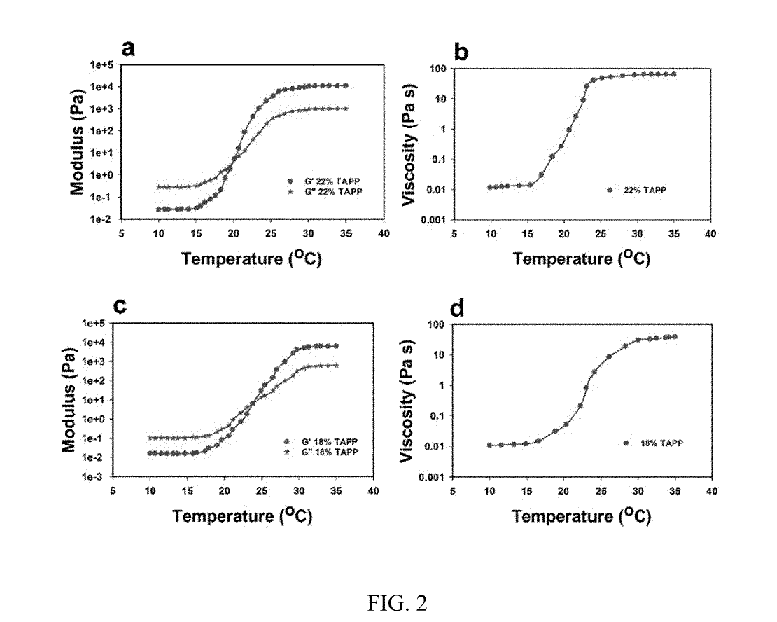

The sol-gel transition process along with the critical micellation temperature was analyzed through dynamic temperature ramp experiments. At low temperatures, the moduli of pluronic-based media were relatively low. It is in fact known that, at low temperatures, pluronic in water solution tends to adopt the form of a unimer. Therefore, low entanglement between the chains would lead to such results. As the temperature was increasing, the breakage of hydrogen bonds and conglomeration of hydrophobic PPO stimulated gelation and led to sharp increases in the moduli (for up to six orders of magnitude). The intersection point between the storage modulus and the loss modulus was considered as the critical micellation temperature (CMT) 23. These points were found to be 20.1° C., 21.8° C. and 23.9° C. respectively for the 22, 20 and 18 weight percent pluronic in TAPP media samples (FIG. 2a). The decrease in CMT with increasing concentrations could be expected because the number of entanglements would increase with increasing concentrations. The viscosity profiles of the pluronic-based media exhibited a behavior similar to that of the moduli during the temperature ramp experiment where the viscosity increases significantly with increasing temperature around the CMT (FIG. 2b). This concurs with reported typical behavior of pluronic, likely due to the fact that with an increase in temperature the chains dehydrate and begin to cross-link leading to closely packed polymeric network. Accordingly, the mesh size of the network decreased with increasing temperature and increasing pluronic concentrations with values ranging from ten to a thousand nanometers.

Microalgal Biomass Production in the TAPP Medium

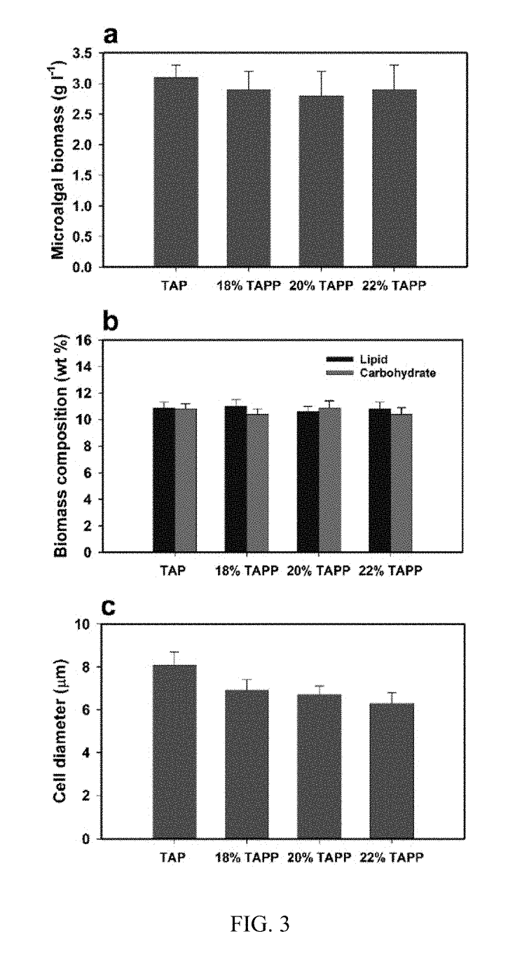

The wild type microalga Chlamydomonas reinhardtii CC-124 was allowed to grow in TAPP media with 3 different pluronic concentrations (18, 20 and 22 weight percent) and a traditional TAP medium culture, used as a control. Therefore, at the temperature of operation (22° C.), the 18% TAPP medium would still be in early micellation stage, the 20% TAPP medium would be close to CMT (i.e. soft gel) and the 22% TAPP sample in final micellation stage. Microalgal growth was assessed under these three conditions through optical density (OD675) analyses and gravimetric measurements. Microalgal biomass concentrations, after a seven-days cultivation period, were found to be 3.1±0.2 g l−1, 2.9±0.3 g l−1, 2.8±0.4 g l−1 and 2.9±0.4 g l−1, respectively for the TAP medium, 18% pluronic, 20% pluronic and the 22% pluronic in TAPP samples (P>0.05) (FIG. 3a). The content in lipid and carbohydrate of the microalgal biomass generated did not vary significantly (P>0.05) under these four conditions (FIG. 3b). These results indicate that microalgae grow well in the TAPP medium with no significant variations in the composition of the generated biomass unlike those commonly seen in other growth medium modification experiments.

The shape and size of microalgal cells confined in TAPP media were assessed through microscopic analyses. While there was no significant change (P>0.05) in the shape of the cells, their size decreased significantly (P<0.05) when growing in the TAPP medium. Average cell diameters were found to be 8.1±0.6 μm, 6.9±0.5 μm, 6.7±0.4 μm and 6.3±0.5 μm, respectively for growth in TAP, 18% pluronic, 20% Pluronic and 22% Pluronic in TAPP samples (FIG. 3c). This decrease in cell size is believed to be the effects of confinement. In fact, at the operating conditions, the three TAPP samples were at different micellation stages and the elastic moduli and mesh sizes exhibited significant differences, which corroborate the observed variation in cell size. Nonetheless, the decrease in cell size does not undermine the fact that the thermoreversible polymer pluronic F-127 can be used in microalgal cultivation since the biomass productivity and composition did not exhibit any significant differences with the control.

Influence of Microalgal Proliferation on the Thermorheological Behavior of the TAPP Medium

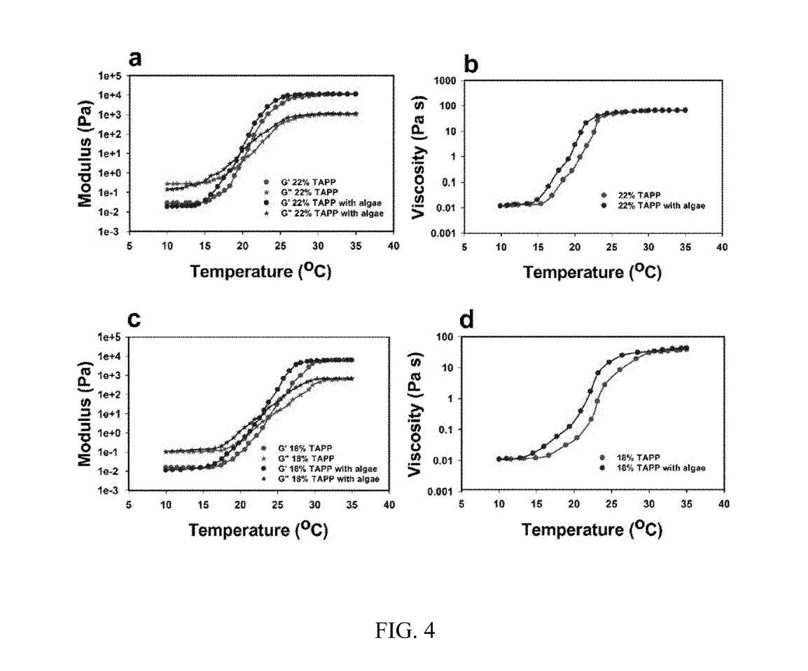

The effects of proliferation of microalgal cells on the thermorheological properties of the TAPP medium were also assessed since large variations on such properties might impact its applicability in microalgal cultivation and harvesting. For this reason, rheological analyses were performed on the resulting microalgae-pluronic matrix after the seven days of microalgal cultivation period. It was observed that, with all three pluronic concentrations TAPP samples (22, 20 and 18 weight percent pluronic), there was a slight decrease in the critical micellation temperature with the presence of microalgal cells. The CMT decreased from 20.1° C. to 19.2° C., 21.8° C. to 20.9° C. and 23.7° C. to 22.8° C. respectively for the 22% pluronic, 20% Pluronic and 18% Pluronic in TAPP samples (FIG. 4a). Similarly, there was a slight shift in the escalation of the viscosity as a response to the increase in temperature (FIG. 4b). This early micellation is likely to be the result of hydrophobic interactions between microalgal cells and pluronic chains accelerating the micellation process. Nonetheless the decrease in the CMT is minor and does not affect the microalgal cultivation and harvesting application.

Harvesting of Microalgae Using Thermoreversible Sol-Gel Transition

One of the major advantages of the TAPP medium is the potential for a simple and efficient microalgal harvesting resulting from the temperature dependent sol-gel transition behavior. The fact that this transition is completely reversible through cooling allows one to control confinement and/or settlement of microalgae through small changes in temperature. As illustrated in the schematic (FIG. 1a), the cultivation and harvesting experiment involved seeding microalgae at a temperature where the TAPP medium is still in solution phase and increasing the temperature beyond (or around) the micellation temperature to allow microalgal cells to grow in a confined environment. After a fixed cultivation period, the temperature was decreased below the critical micellation temperature and microalgae settled at the bottom. Afterwards, the temperature was increased to jellify the supernatant and harvesting of microalgae simply involved scraping microalgal flocs off the surface.

The distribution of microalgal cells within the TAPP medium was an important parameter for the proposed harvesting system. We hypothesized that under the selected cultivation conditions microalgal cells would be distributed in clusters due to confinement as opposed to randomly distributed cells in the traditional TAP medium. There was an interest to characterize these clusters because their morphology would impact the velocity to which they settle with faster settling for spherically shaped clusters. Furthermore, there is a direct correlation between the size of the clusters and the settling velocity, both according to Stokes' law or the empirical formulas often used to determine settling velocity beyond the Stokes regime.

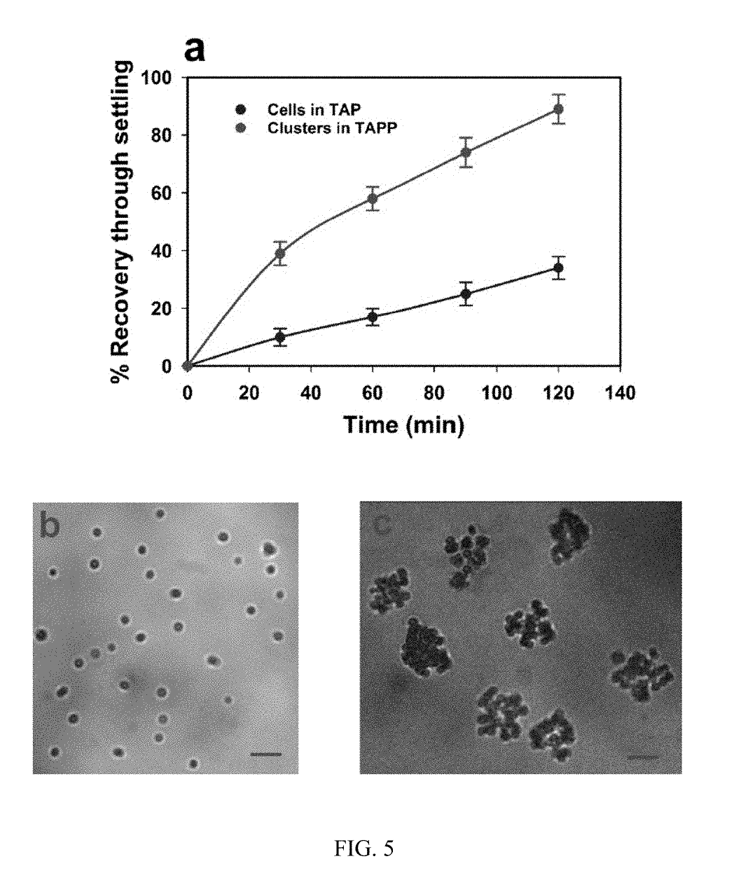

The distribution of microalgal cells were assessed through random selection of images captured with an Axio Imager M1 microscope (Carl Zeiss Inc., Berlin, Germany) on each batch of microalgal culture. The images where then processed and the shape and size of cells and clusters were characterized with a Zen pro software (Carl Zeiss Inc., Berlin, Germany). As predicted, microalgal cells from the TAPP system were observed as regrouped in clusters (FIG. 5c) whereas those from the control (TAP medium) were visualized as randomly dispersed cells (FIG. 5b). The average form factor of the microalgal clusters concurred with a sphericity approximation with a value of 0.98±0.02 μm which justifies the use of Stokes' law to predict the settling velocity. The average equivalent diameter of the clusters was found to be 78±9 μm compared to an 8.1±0.6 μm average diameter for isolated cells in TAP medium. Considering the viscosity measured during the rheological characterization, the settling velocity was calculated according to Stokes' law and averaged a value of 2.6±6 m day-1 which is about ten times greater than the estimated settling velocity of isolated microalgal cells in TAP medium (0.27±0.03 m day-1). To verify the settling rate experimentally, microalgae were allowed to settle at 15° C. in tubes with 10 cm working height after the seven days microalgal cultivation period. The optical density (OD675) of the TAPP broth was measured at regular intervals and the variation in microalgal biomass concentration was compared against a TAP broth used as control. The optical density measurements during the settling assay were used to compute the percent recovery through settling over time. It was found that over a 2-hour period, 89±5% of microalgal clusters were recovered through settling compared to 34±3% for isolated cells in TAP for the 10 cm working height (FIG. 5a). It is clear that these experimental results exhibited slower biomass recovery compared to the predictions using Stokes' law. Similar deviations of experimental data from predicted settling rate are often reported and may be due to the large heterogeneity of cluster and cell sizes. Nonetheless there is a corroboration for a largely higher settling velocity for microalgal clusters in TAPP medium compared to settling of dispersed microalgal cells in TAP medium.

To evaluate the harvesting efficiency through scraping of microalgae off the surface of the TAPP system, two-capped containers may be used or one-capped containers may be flipped upside down before the decrease in temperature for clusters settling. The percentage of microalgae harvested was assessed through gravimetric measurements on the harvested biomass and also through optical density measurements on the remaining broth. The harvesting efficiency was then computed as the percent difference between microalgal concentrations of the broths prior and after harvesting. It was found that 89±2%, 88±3% and 86±2% of microalgae were harvested respectively for 22% pluronic, 20% Pluronic and 18% Pluronic in TAPP samples.

Methods

TAPP Medium Preparation and Culture Conditions

Pluronic F-127 was obtained from BASF (Ludwigshafen, Germany) and was used without further purification. The chemical composition for this pluronic type is PEO₁₀₀PPO₆₅PEO₁₀₀ and the total molecular weight is 12600 g mol⁻¹. PEO and PPO ratio is approximately 2:1 by weight. The pluronic-based growth medium was prepared in a way that maintains a concentration of nutrients similar to the traditional Tris-Acetate-Phosphate (TAP) medium with the addition of pluronic (TAPP medium) that confers the thermoreversible sol-gel transition properties. The concentrations of chemicals in the TAPP medium were therefore as follow: Tris (19.98 mM), NH₄Cl (70.11 mM), MgSO₄.7H2O (4.06 mM), CaCl₂.2H₂O (3.40 mM), K₂HPO₄.3H₂O (0.47 mM), KH₂PO₄ (0.40 mM), acetic acid (0.1% vol), Hutner's trace (0.1% vol). Pluronic F-127 powder was dissolved in the medium at 4° C. for 5 hours and under vigorous stirring. Final concentrations of pluronic in TAPP media were selected to be 18%, 20% and 22% (weight percent) in order to obtain a range of CMT suitable for the microalgal cultivation and harvesting application.

Cultivation of wild type microalgae Chlamydomonas reinhardtii CC-124, obtained from the Chlamydomonas Resource Center (University of Minnesota, St. Paul, Minn.), was performed in the TAPP medium. Aliquots of 0.5 ml from liquid subcultures prepared five days preceding the experiment were mixed with 50 ml of the TAPP medium at 15° C. (below the CMT of the samples). Subsequently, vials containing microalgal culture were placed on a rotary shaker in a room continuously illuminated by full spectrum compact fluorescent lamps (CFL 60W, Fancierstudio, San Francisco, Calif.) with the photosynthetic active radiation at the top surface of the culture at 100±5 μE m−2 s−1 and the temperature at 22±1° C. After seven days of cultivation, the resulting Pluronic-microalgae matrix was used for thermorheological characterization and biomass production analyses.

Rheometry

Rheological experiments to characterize the properties of the TAPP medium were performed using a Combined Motor and Transducer (CMT) AR-G2 rheometer from TA instruments (New Castle, Del.). The cone-and-plate geometry with a diameter of 40 mm and a cone angle of 0° 59′ 49″ was used for all the measurements. The temperature control was achieved by a Peltier plate using thermoelectric effects to control the temperature accurately and water circulation for rapid heating and cooling over a temperature range of 0 to 100° C.

Characterization of Microalgal Biomass Production and Harvesting

The effects of pluronic presence on microalgal cultivation and biomass production were assessed using different analytical techniques. Microalgal biomass production was evaluated through optical density measurements and gravimetric quantification. Microalgal biomass carbohydrate content was assessed using the phenol-sulfuric acid method and lipids content through a modified Bligh and Dyer method and fluorescence scanning using Nile Red dye. The impacts of the pluronic-based environment on shape and size of microalgal cells were systematically analyzed using an Axio Imager M1 microscope and a ZEN pro software (Carl Zeiss Inc., Berlin, Germany). These same tools were also used to characterize distribution of microalgal cells (clusters etc.) in TAPP media with different pluronic concentrations as well as the traditional TAP medium used as control. The shape and size of microalgal clusters were characterized in order to predict the settling velocity for harvesting. The form factor (FF) characterizing the deviation from a circle and the equivalent diameter (De) of microalgal clusters were computed as presented by Grijspeerdt and Verstraete:

$$\begin{matrix}FF = \frac{4 * π * Area}{{Perimeter}^{2}} & ( 1 ) \\ De = 2 * \sqrt{\frac{Area}{π}} & ( 2 )\end{matrix}$$

The settling velocity (V) of microalgal clusters during the harvesting process could be approximated using Stokes' law as long as the form factor concurred with a spherical shape and the Reynold number fell within the Stokes' regime. Under such conditions the settling velocity was calculated as:

$$V = \frac{( ρ s - ρ l ) * g * {De}^{2}}{18 μ} \qquad ( 3 )$$

Where ρs and μl are solid and liquid densities, g the gravitational acceleration, De the equivalent diameter and μ the dynamic viscosity. The average settling velocity was then experimentally monitored by allowing microalgae to settle at 15° C. in columns with 10 cm working height and taking regular optical density measurements (OD675) on the broths. To estimate the rate of settlement, the percent recovery at each measurement time was computed as follows:

$$\% Recovery = ( 1 - \frac{{OD }_{675} ( t )}{{OD }_{675} ( to )} ) * 100 \qquad ( 3 )$$

Where OD₆₇₅(t) is the optical density of the broth after a settling time (t) and OD₆₇₅(to) the optical density at the beginning of the settling experiment or time (to).

Statistical Analyses

The microalgal cultivation and harvesting experiments and the related biochemical and rheological analyses mentioned previously were repeated at least 10 times with triplicate measurements for each run. Statistical analyses over the data collected were performed using Minitab software. The results with P-Value less than 0.05 (t-test) were considered statistically significant.

Another advantage of the TAPP medium is the potential for recycling and reuse of the medium for recultivation of microalgae after the harvesting process. The thermoreversible sol-gel transition properties of the TAPP medium are not altered after cultivation and harvesting of microalgae. Therefore, recultivation of microalgae in the recycled TAPP medium simply requires a replenishment of nutrients based on the nutrient uptake in the preceding microalgal culture32-34. This was confirmed by recycling and reusing the TAPP medium for the recultivation of microalgae in a three cultivation cycles experiment. After each cultivation cycle, the microalgal biomass was quantified and harvested and the TAPP growth medium was recycled and reused for another cultivation. Starting with the same initial biomass concentration in each microalgal cultivation cycle, final microalgal biomass concentrations were found to be 2.8±0.2 g l⁻¹, 2.3±0.3 g l⁻¹ and 2.5±0.3 g l⁻¹ respectively for the first, second and third cultivation cycles.

What is claimed is:

1. A culture medium, comprising:
a Tris-Acetate-Phosphate solution; and
an amount of pluronic dissolved in the Tris-Acetate-Phosphate solution.

2. The medium of claim 1, wherein the amount of pluronic dissolved in the Tris-Acetate-Phosphate solution results in a concentration of pluronic of at least 18 percent by weight.

3. The medium of claim 2, wherein the amount of pluronic dissolved in the Tris-Acetate-Phosphate solution results in a concentration of pluronic of at least 20 percent by weight.

4. The medium of claim 3, wherein the amount of pluronic dissolved in the Tris-Acetate-Phosphate solution results in a concentration of pluronic of at least 22 percent by weight.

5. The medium of claim 3, wherein the chemical composition of the pluronic is PEO₁₀₀PPO₆₅PEO₁₀₀ and the total molecular weight is 12600 g mol⁻¹.

6. The medium of claim 5, wherein the pluronic has a ratio of PEO to PPO of 2:1 by weight.

7. A method of culturing an organism, comprising the steps of:
providing a culture medium comprising a Tris-Acetate-Phosphate solution and an amount of pluronic dissolved in the Tris-Acetate-Phosphate solution;
maintaining the culture medium at a first temperature where the culture medium is in a sol state;
seeding the culture medium with an organism to be cultured;
heating the culture medium to a second temperature where the culture medium is in a gel state;
allowing the organism to grow while the culture medium is in the gel state.

8. The method of claim 7, further comprising the step of cooling the culture medium to a third temperature where the culture medium is in a sol state.

9. The method of claim 8, further comprising the step of allowing the organism to settle with the culture medium is in a sol state.

10. The method of claim 9, further comprising the step of heating the culture medium to a fourth temperature where the culture medium is in a sol state.

11. The method of claim 10, further comprising the step of harvesting the organism that has settled from the culture medium while it is in a gel state.

12. The method of claim 11, wherein the first temperature is below a sol to gel transition temperature of the culture medium.

13. The method of claim 11, wherein the second temperature is above the sol-gel transition temperature of the culture medium.

14. The method of claim 11, wherein the third temperature is below the sol-gel transition temperature of the culture medium.

15. The method of claim 11, wherein the fourth temperature is above the sol-gel transition temperature of the culture medium.
